(19) United States
(12) Patent Application Publication
(10) Pub. No.: US 2023/0256417 A1
(43) Pub. Date: Aug. 17, 2023

(54) PHOTOCATALYST-ATTACHED FILTER AND PREPARING METHOD OF THE SAME

(71) Applicants: RESEARCH & BUSINESS FOUNDATION SUNGKYUNKWAN UNIVERSITY, Suwon-si (KR); INSTITUTE FOR BASIC SCIENCE, Daejeon (KR)

(72) Inventors: Hyoyoung Lee, Uiwang-si (KR); Jung Sue CHOI, Suwon-si (KR); Hak Sung LEE, Suwon-si (KR); Yeon Su HAN, Suwon-si (KR); Tae Hun YANG, Suwon-si (KR); Hyun KO, Suwon-si (KR)

(73) Assignees: RESEARCH & BUSINESS FOUNDATION SUNGKYUNKWAN UNIVERSITY, Suwon-si (KR); INSTITUTE FOR BASIC SCIENCE, Daejeon (KR)

(21) Appl. No.: 18/110,028

(22) Filed: Feb. 15, 2023

(30) Foreign Application Priority Data
Feb. 15, 2022 (KR) 10-2022-0019296

(51) Int. Cl. B01J 23/30; B01J 21/06; B01J 31/06; B01J 31/38; B01J 35/00; B01J 37/04; B01J 37/00; B01J 37/02; A61L 9/20; B01D 53/86; B01D 53/00; B01D 53/88

(52) U.S. Cl. CPC B01J 23/30; B01J 21/063; B01J 31/061; B01J 31/38; B01J 35/0013; B01J 37/04; B01J 37/0018; B01J 37/0219; B01J 37/0217; A61L 9/20; B01D 53/8668; B01D 53/007; B01D 53/885; A61L 2209/12; A61L 2209/14; B01D 2255/20707; B01D 2255/20776; B01D 2255/802; B01D 2257/708; B01D 2259/804; B01D 2255/915

(57) ABSTRACT

The present application relates to a filter having a photocatalyst attached thereto, which comprises: a substrate; and a photocatalyst bonded on the substrate, in which the photocatalyst has each photocatalyst bonded and combined by a polymer binder, and the substrate and the photocatalyst are bonded by a hydrophilic polymer binder.

### BACKGROUND OF THE INVENTION

### 1. Field of the Invention

The present application relates to a filter having a photocatalyst attached thereto and a method for manufacturing the same.

### 2. Description of the Prior Art

Air pollution and environmental contamination caused by technological advance and development, and industrialization have been recognized as major problems worldwide, and various heavy metals or chemicals included in exhaust fumes from factories and automobiles have been faced as a big issue to be solved. Among those heavy metals or chemicals, volatile organic compounds contained in soot, fine dust and the like are extremely harmful not only to the environment but also to the human body, and many studies are being conducted to overcome the problem, and many companies are also working to develop technologies or products capable of addressing these compounds.

A high efficiency particulate air filter (HEPA) is a commercially available air purifying product. HEPA filters usually have a network structure made of organic polymers, and have the ability to collect about 85% to 99.975% of particles having a size of 0.3 μm depending on grades. The HEPA filters collect particles such as fine dust, etc., by using interception by fiber structure, particle sedimentation by collision and gravity, adsorption by Brownian motion of particles and electrostatic force, etc., according to particle sizes, but do not decompose the collected particles. In addition, there is a problem in that the filters cannot collect, remove, or decompose volatile organic compounds, including carcinogens which directly or indirectly threaten our lives.

In order to solve these problems, research is being conducted on a photocatalytic filter which adsorbs and decomposes harmful substances in the air. In general, the photocatalyst filter uses a binder to coat the photocatalyst on the filter, and a silicon alkoxide such as tetraethyl orthosilicate (TEOS), a fluorine-based resin such as polytetrafluoroethylene, or a hydrophobic material such as epoxy, etc., are used as a binder. However, when using such a hydrophobic binder, there is also a disadvantage in that the photocatalyst has a reduced area coming into contact with the outside, resulting in less efficiency, and is not environmentally friendly.

Therefore, the efficiency of the photocatalyst is not reduced when coated on the filter, and there is a need for the development of a filter having a photocatalyst attached thereto, which has moisture resistance, heat resistance, impact resistance, abrasion resistance, water resistance, acid resistance, and the like.

Korean Unexamined Patent Publication No. 10-2021-0080854 relates to an air purifier for vehicles using a photocatalyst filter which acts in a visible light region. The above patent describes a photocatalyst filter having a photocatalyst coated on the surface of a metal mesh, but uses tetraethyl orthosilicate (TEOS), which is a hydrophobic binder, as a binder for coating the photocatalyst, and does not mention about coating the photocatalyst with a hydrophilic binder.

### SUMMARY OF THE INVENTION

To solve the aforementioned problems of the related art, an object of the present application is to provide a filter having a photocatalyst attached thereto.

In addition, an object of the present application is to provide a method for manufacturing the filter having the photocatalyst attached thereto.

Furthermore, an object of the present application is to provide an air purifying device including the filter having the photocatalyst attached thereto.

However, the technical problems to be achieved by the embodiments of the present application are not limited to the technical problems described above, and other technical problems may exist.

As a technical solution for achieving the above technical problems, a first aspect of the present application may provide a filter having a photocatalyst attached thereto, which includes: a substrate; and a photocatalyst bonded on the substrate, in which the photocatalyst has each photocatalyst bonded and combined by a polymer binder, and the substrate and the photocatalyst are bonded by the polymer binder.

According to one embodiment of the present application, the polymer binder may include a hydrophilic polymer, but is not limited thereto.

According to one embodiment of the present application, the hydrophilic polymer may include one selected from the group consisting of carboxymethyl cellulose (CMC), polyvinyl alcohol (PVA), polyvinylidene fluoride (PVDF), polytetrafluoroethylene (PTFE), polyamide, polyamine, styrene-butadiene rubber, and combinations thereof, but is not limited thereto.

According to one embodiment of the present application, the photocatalyst may include an anatase phase and a rutile phase, and may include reduced titanium dioxide in which one of the anatase phase and the rutile phase is selectively reduced, but is not limited thereto.

According to one embodiment of the present application, the reduced titanium dioxide may be in the form of blue nanoparticles, but is not limited thereto.

According to one embodiment of the present application, the photocatalyst may further include one selected from the group consisting of WO₃, TiO₂ (anatase), TiO₂ (rutile), TiO₂, in which an anatase phase and a rutile phase are mixed, ZnO, CdS, ZrO₂, SnO₂, V₂O₃, and combinations thereof, but is not limited thereto.

According to one embodiment of the present application, the substrate may include one selected from the group consisting of polyethylene terephthalate, polyethylene, polypropylene, polycarbonate, polyvinyl chloride, polystyrene, concrete, glass, ceramic, metal, paper, wood, and combinations thereof, but is not limited thereto.

In addition, a second aspect of the present application may provide a method for manufacturing a filter having a photocatalyst attached thereto, which includes: dispersing a solution containing a photocatalyst and a polymer binder; dropping the solution onto a substrate; and cooling the substrate.

According to one embodiment of the present application, the substrate may be heated before performing the dropping, or may be heated after performing the dropping, but is not limited thereto.

According to one embodiment of the present application, when the substrate is a polymer compound, the heating may be performed at or above a glass transition temperature of the polymer compound, but is not limited thereto.

According to one embodiment of the present application, the substrate heated at or above the glass transition temperature may have the photocatalyst bonded by the cooling, but is not limited thereto.

According to one embodiment of the present application, the polymer binder may include a hydrophilic polymer, but is not limited thereto.

According to one embodiment of the present application, the hydrophilic polymer may include one selected from the group consisting of carboxymethyl cellulose (CMC), polyvinyl alcohol (PVA), polyvinylidene fluoride (PVDF), polytetrafluoroethylene (PTFE), polyamide, polyamine, styrene-butadiene rubber, and combinations thereof, but is not limited thereto.

According to one embodiment of the present application, the dispersion may be performed in a process selected from the group consisting of an ultrasonic process, a pulverization process by physical impact force, a pulverization process by physical shear force, a high-pressure process, a supercritical/subcritical process, and combinations thereof, but is not limited thereto.

According to one embodiment of the present application, the dropping may be performed by using one selected from the group consisting of a dropper, a spray gun, and combinations thereof, but is not limited thereto.

According to one embodiment of the present application, the photocatalyst may include an anatase phase and a rutile phase, and may include reduced titanium dioxide in which one of the anatase phase and the rutile phase is selectively reduced, but is not limited thereto.

According to one embodiment of the present application, the photocatalyst may further include one selected from the group consisting of WO₃, TiO₂ (anatase), TiO₂ (rutile), TiO₂, in which an anatase phase and a rutile phase are mixed, ZnO, CdS, ZrO₂, SnO₂, V₂O₃, and combinations thereof, but is not limited thereto.

In addition, a third aspect of the present application may provide an air purifying device including a filter having a photocatalyst attached thereto according to a first aspect of the present application.

The above-described technical solutions are set forth to illustrate only and should not be construed as intended to limit the present disclosure. In addition to the exemplary embodiments described above, additional embodiments may exist in the drawings and detailed description of the invention.

A conventional filter having a photocatalyst attached thereto uses a hydrophobic binder to apply and coat the catalyst on a substrate, but as the hydrophobic binder is used, there has been a problem in that the photocatalyst has a reduced area coming into contact with the outside, resulting in less decomposition efficiency of organic compounds, and is not environmentally friendly.

Meanwhile, according to the present application, there may be provided a filter having a photocatalyst attached thereto, which has the photocatalyst bonded to the filter by using a hydrophilic polymer binder, has a strong binding effect by using the hydrophilic polymer binder, as well as strong moisture resistance, heat resistance, impact resistance, abrasion resistance, and water resistance by using the hydrophilic polymer binder, and has improved decomposition efficiency of volatile organic compounds.

In addition, when the substrate used in manufacturing is a polymer compound, the filter having the photocatalyst attached thereto can have the photocatalyst strongly bonded to the substrate through a process of dropping a solution containing a photocatalyst, when the substrate is heated at or above a glass transition temperature and a phase change from a solid phase to a liquid phase occurs, or dropping a solution containing a photocatalyst on a substrate, then heating the substrate to a glass transition temperature or higher so as to induce a phase change from a solid phase to a liquid phase, and then cooling the polymer below a glass transition temperature so as to change the same into a solid phase.

Furthermore, the filter having the photocatalyst attached thereto according to the present application can be manufactured by using a substrate made of various materials such as metal, paper, wood, concrete, glass, ceramic, etc., as well as a polymer compound as a substrate by using a hydrophilic polymer binder.

However, the effects obtainable herein are not limited to the effects described above, and other effects may also exist.

### BRIEF DESCRIPTION OF THE DRAWINGS

FIG. 1 is a schematic view of reactions occurring on a photocatalyst, a polymer binder, and the surface of a substrate according to one embodiment of the present application.

FIG. 2 is a flow chart of a method for preparing a catalyst having a photocatalyst attached thereto according to one embodiment of the present application.

FIG. 3 is a digital camera picture and an optical microscope picture of filters according to one example and a comparative example of the present application.

FIG. 4 is an SEM image of filters according to one example and a comparative example of the present application.

FIG. 5 is an SEM-EDS mapping image of filters according to one example of the present application.

FIG. 6 is a Raman spectroscopic spectrum image of filters according to one example and a comparative example of the present application.

FIG. 7 is an X-ray diffraction spectroscopic spectrum image of filters according to one example and a comparative example of the present application.

(A) of FIG. 8 is an ultraviolet-visible ray absorbance spectrum image of filters according to one example and a comparative example of the present application, (B) thereof is a graph of measuring an acetaldehyde reduction effect.

FIG. 9 is a schematic view of an air purifying device to which a filter according to one example of the present application is applied.

FIG. 10 is an SEM image of filters according to one example and a comparative example of the present application.

FIG. 11A and FIG. 11B are EDS analysis data of filters according to one example and a comparative example of the present application.

FIG. 12 is an experimental picture of comparing water resistance of filters according to an example and a comparative example of the present application.

FIG. 13 is an UV/VIS absorption spectrum image of filters according to one example and a comparative example of the present application.

FIG. 14 is a graph for explaining catalytic efficiency of a photocatalyst powder according to one example of the present application.

### DETAILED DESCRIPTION OF THE EXEMPLARY EMBODIMENTS

With reference to the accompanying drawings, embodiments of the present application will be described in detail as follows such that those skilled in the art to which the present application pertains may easily practice the present application.

However, the present application may be implemented in various different forms, and is not limited to the embodiments described herein. And, in order to clearly describe the present application in the drawings, parts irrelevant to the description may be omitted, and similar reference numerals may be attached to similar parts throughout the specification.

Throughout present specification, when a part is said to be “connected” to another part, this may include not only the case of being “directly connected” but also the case of being “electrically connected” with another element interposed therebetween.

Throughout present specification, when a member is referred to as being “on,” “at the upper portion of,” “at the top of,” “under,” “at the lower portion of,” or “at the bottom of” another member, this may include not only the case where a member is in contact with another member, but also the case where another member exists between two members.

Throughout present specification, when any part is said to “include” a certain component, this means that the part may further include other components rather than excluding the other components, unless otherwise particularly specified.

As used herein, the terms “about,” “substantially,” and the like may be used in a sense at or close to that number when manufacturing and material tolerances inherent in the stated meaning are given, and may be also used to prevent unfair use by unscrupulous infringers of disclosures in which exact or absolute figures are stated to aid in the understanding of the present application. In addition, throughout the present specification, “steps of doing” or “steps of” may not mean “steps for.”

Throughout the present specification, the term “combination thereof” included in the expression of the Markush form may means one or more mixtures or combinations selected from the group consisting of the components described in the expression of the Markush form, and may mean including one or more selected from the group consisting of the components.

Throughout present specification, reference to “A and/or B” may mean “A or B, or A and B.”

Hereinafter, the filter having the photocatalyst attached thereto according to the present application and the method for manufacturing the same may be described in detail with reference to embodiments and examples and drawings. However, the present application is not limited to these embodiments and examples and drawings.

As a technical solution for achieving the above technical problems, a first aspect of the present application may provide a filter having a photocatalyst attached thereto, which includes: a substrate; and a photocatalyst bonded on the substrate, in which the photocatalyst has each photocatalyst bonded and combined by a polymer binder, and the substrate and the photocatalyst are bonded by the hydrophilic polymer binder.

According to one embodiment of the present application, the polymer binder may include a hydrophilic polymer, but is not limited thereto.

A conventional filter having a photocatalyst attached thereto uses a hydrophobic binder to apply and coat the catalyst on a substrate, but as the hydrophobic binder is used, there has been a problem in that the photocatalyst has a reduced area coming into contact with the outside, resulting in less decomposition efficiency of organic compounds, and is not environmentally friendly.

Meanwhile, according to the present application, there may be provided a filter having a photocatalyst attached thereto, which has the photocatalyst bonded to the filter by using a hydrophilic polymer binder, has a strong binding effect by using the hydrophilic polymer binder, as well as strong moisture resistance, heat resistance, impact resistance, abrasion resistance, and water resistance by using the hydrophilic polymer binder, and has improved decomposition efficiency of volatile organic compounds.

In addition, when the substrate used in manufacturing is a polymer compound, the filter having the photocatalyst attached thereto can have the photocatalyst strongly bonded to the substrate through a process of dropping a solution containing a photocatalyst, when the substrate is heated at or above a glass transition temperature and a phase change from a solid phase to a liquid phase occurs, or dropping a solution containing a photocatalyst on a substrate, then heating the substrate to a glass transition temperature or higher so as to induce a phase change from a solid phase to a liquid phase, and then cooling the polymer below a glass transition temperature so as to change the same into a solid phase.

Furthermore, the filter having the photocatalyst attached thereto according to the present application can be manufactured by using a substrate made of various materials such as metal, paper, wood, concrete, glass, ceramic, etc., as well as a polymer compound as a substrate by using a hydrophilic polymer binder.

According to one embodiment of the present application, the hydrophilic polymer may include one selected from the group consisting of carboxymethyl cellulose (CMC), polyvinyl alcohol (PVA), polyvinylidene fluoride (PVDF), polytetrafluoroethylene (PTFE), polyamide, polyamine, styrene-butadiene rubber, and combinations thereof, but is not limited thereto.

By including the hydrophilic polymer as the polymer binder, the photocatalyst may be strongly bonded to the substrate, thus providing an effect of moisture resistance, heat resistance, impact resistance, and abrasion resistance may be provided, and improving decomposition efficiency of volatile organic compounds.

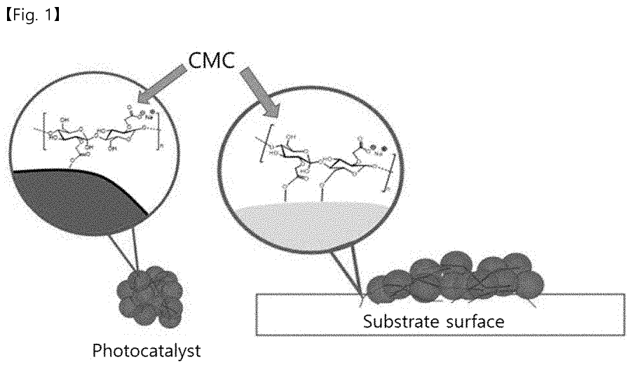

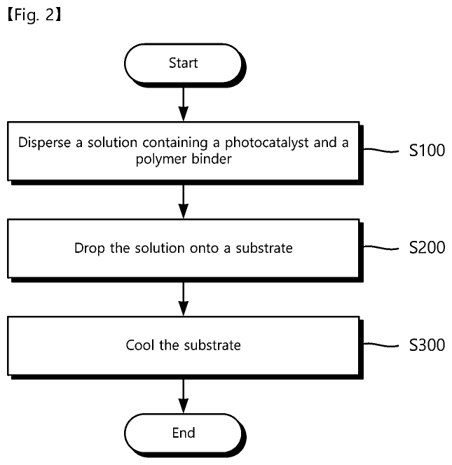

FIG. 1 is a schematic view of reactions occurring on a photocatalyst, a polymer binder, and the surface of a substrate according to one embodiment of the present application. Specifically, FIG. 1 is a schematic view of reactions when using carboxymethyl cellulose (CMC), a hydrophilic polymer, as a polymer binder.

Referring to FIG. 1, it can be confirmed that the carboxyl group or hydroxyl group of CMC strongly binds the substrate and photocatalyst through a dehydration condensation reaction with the hydroxyl group on the surface of the photocatalyst or the hydroxyl group on the substrate surface, and it can be seen that the photocatalyst may be bonded to a material or a substrate having a carboxyl group or a hydroxyl group by using a hydrophilic polymer as a polymer binder.

According to one embodiment of the present application, the photocatalyst may include an anatase phase and a rutile phase, and may include reduced titanium dioxide in which one of the anatase phase and the rutile phase is selectively reduced, but is not limited thereto.

Titanium dioxide existing in nature may largely include two phases of an anatase phase and/or a rutile phase, and physical properties may change due to various reasons such as the ratio of the two phases, etc. Generally, the band gap of the titanium dioxide may be about 3.1 eV.

The rutile phase according to the present application may be also known as rutile, and most of titanium dioxide in nature may have a rutile phase. The rutile phase may be superior to the anatase phase in weather resistance, hiding power, white brightness, dielectric constant, and the like.

The anatase phase according to the present application may have excellent photocatalytic activity for decomposing contaminants present in water or air, and may have wear resistance improved when titanium dioxide of the anatase phase is coated on other materials.

When light is irradiated on titanium dioxide containing the rutile phase and/or the anatase phase, it may be used for various purposes such as photocatalyst, solar cell, organic material removal, etc. However, when titanium dioxide in a natural state is simply used without any process, there are disadvantages in that commercialization is difficult due to relatively low efficiency, reaction only to light of a specific wavelength, and the like.

The filter having the photocatalyst attached thereto according to the present application may include the reduced titanium dioxide, which means a material, in which at least one of the rutile phase and the anatase phase is reduced, and the other is not reduced. For example, the reduced titanium dioxide may include a reduced rutile phase and an unreduced anatase phase, or may include a reduced anatase phase and an unreduced rutile phase, but is not limited thereto.

According to one embodiment of the present application, the reduced titanium dioxide may be in the form of blue nanoparticles, but is not limited thereto.

According to one embodiment of the present application, the photocatalyst may further include one selected from the group consisting of WO₃, TiO₂ (anatase), TiO₂ (rutile), TiO₂, in which an anatase phase and a rutile phase are mixed, ZnO, CdS, ZrO₂, SnO₂, V₂O₃, and combinations thereof, but is not limited thereto.

The filter having the photocatalyst attached thereto according to the present application may further use a commonly used photocatalyst (for example, WO₃, etc.) in addition to the reduced titanium dioxide as a photocatalyst, thereby providing a filter to which two or more types of photocatalysts are attached. In the case of the reduced titanium dioxide, which are used with WO₃, excited electrons generated in the conduction band of WO₃ may move to trap holes in the valance band of reduced titanium dioxide by a Z scheme. After the separation, the excited electrons moved to the conduction band of titanium dioxide may reduce harmful substances such as VOC more efficiently than used as a single substance. Accordingly, this case may provide a higher harmful substance decomposition efficiency than when using a single photocatalyst, but is not limited thereto.

According to one embodiment of the present application, the substrate may include one selected from the group consisting of polyethylene terephthalate, polyethylene, polypropylene, polycarbonate, polyvinyl chloride, polystyrene, concrete, glass, ceramic, metal, paper, wood, and combinations thereof, but is not limited thereto.

The filter having the photocatalyst attached thereto according to the present application may use a polymer compound (for example, polyethylene terephthalate, etc.) used in general filters as a substrate by using a hydrophilic polymer as the polymer binder, so that the photocatalyst may be attached thereto, and the photocatalyst may be attached to substrates made of various materials, such as concrete, glass, ceramics, metal, paper, wood, etc., in addition to polymer compound.

In addition, a second aspect of the present application may provide a method for manufacturing a filter having a photocatalyst attached thereto, which includes: dispersing a solution containing a photocatalyst and a polymer binder; dropping the solution onto a substrate; and cooling the substrate.

With respect to the method for manufacturing the filter having the photocatalyst attached thereto according to the second aspect of the present application, detailed descriptions of parts overlapping with the first aspect of the present application have been omitted, but even if the description is omitted, the contents described in the first aspect of the present application may be applied to the second aspect of the present application.

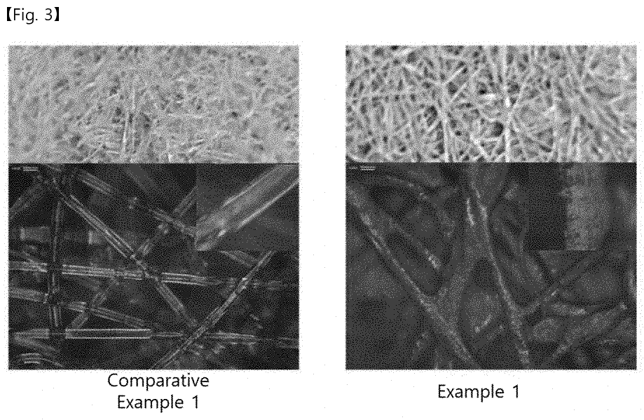

FIG. 2 is a flow chart of a method for preparing a catalyst having a photocatalyst attached thereto according to one embodiment of the present application.

First, a solution containing a photocatalyst and a polymer binder may be dispersed (S100).

According to one embodiment of the present application, the polymer binder may include a hydrophilic polymer, but is not limited thereto.

According to one embodiment of the present application, the hydrophilic polymer may include one selected from the group consisting of carboxymethyl cellulose (CMC), polyvinyl alcohol (PVA), polyvinylidene fluoride (PVDF), polytetrafluoroethylene (PTFE), polyamide, polyamine, styrene-butadiene rubber, and combinations thereof, but is not limited thereto.

According to one embodiment of the present application, the photocatalyst may include an anatase phase and a rutile phase, and may include reduced titanium dioxide in which one of the anatase phase and the rutile phase is selectively reduced, but is not limited thereto.

According to one embodiment of the present application, the photocatalyst may further include one selected from the group consisting of WO₃, TiO₂ (anatase), TiO₂ (rutile), TiO₂, in which an anatase phase and a rutile phase are mixed, ZnO, CdS, ZrO₂, SnO₂, V₂O₃, and combinations thereof, but is not limited thereto.

According to one embodiment of the present application, the dispersion may be performed in a process selected from the group consisting of an ultrasonic process, a pulverization process by physical impact force, a pulverization process by physical shear force, a high-pressure process, a supercritical/subcritical process, and combinations thereof, but is not limited thereto.

Then, the solution may be dropped on the substrate (S200).

According to one embodiment of the present application, the substrate may be heated before performing the dropping, or may be heated after performing the dropping, but is not limited thereto.

According to one embodiment of the present application, when the substrate is a polymer compound, the heating may be performed at or above a glass transition temperature of the polymer compound, but is not limited thereto.

When the substrate used in manufacturing is a polymer compound, the filter having the photocatalyst attached thereto may have the photocatalyst strongly bonded to the substrate through a process of dropping a solution containing a photocatalyst, when the substrate is heated at or above a glass transition temperature and a phase change from a solid phase to a liquid phase occurs, or dropping a solution containing a photocatalyst on a substrate, then heating the substrate to a glass transition temperature or higher so as to induce a phase change from a solid phase to a liquid phase, and then cooling the polymer below a glass transition temperature so as to change the same into a solid phase.

Specifically, when a polymer compound used as the substrate is heated at a glass transition temperature or higher to cause a phase change of the polymer compound from a solid phase to a liquid phase, the photocatalyst may be bound with a property similar to a behavior of an adhesive by a polymer compound which has started to change into a liquid phase. This may not be a chemical bond due to a specific functional group, and thus may serve as a method applicable to all organic polymers having a glass transition temperature. At this time, any material which is stable up to about 100° C. as well as metal oxide may be used as the photocatalyst.

According to one embodiment of the present application, the dropping may be performed by using one selected from the group consisting of a dropper, a spray gun, and combinations thereof, but is not limited thereto.

According to one example, the substrate may be a HEPA filter. Before dropping the solution, the HEPA filter may be immersed in water and ultrasonically washed, and then dried in a vacuum oven.

In addition, according to one example, the substrate may include be hydrophilic material. Specifically, as described above, when the polymer binder is the hydrophilic polymer (for example, carboxymethylcellulose), it may be easily attached to the substrate of the hydrophilic material by the hydroxyl group of the hydrophilic polymer.

Lastly, the substrate may be cooled (S300).

According to one embodiment of the present application, the substrate heated at or above the glass transition temperature may have the photocatalyst bonded by the cooling, but is not limited thereto.

When the substrate is a polymer compound, the photocatalyst may be strongly bonded to the substrate through a process of cooling the substrate at or below the glass transition temperature of the polymer compound, so as to change the same into a solid phase.

In addition, a third aspect of the present application may provide an air purifying device including a filter having a photocatalyst attached thereto according to a first aspect of the present application.

With respect to the air purifying device according to the third aspect of the present application, detailed descriptions of parts overlapping with the first and/or second aspects of the present application have been omitted, but even if the description is omitted, the contents described in the first and/or second aspects of the present application may be applied to the third aspect of the present application.

Hereinafter, the present invention will be described in more detail through the following examples, but the following examples are for illustrative purposes only and are not intended to limit the scope of the present application.

### [Preparation Example] Preparation of Reduced Titanium Dioxide (Blue TiO₂) Photocatalyst

A titanium dioxide (Blue TiO₂) photocatalyst, in which only one of the anatase phase and the rutile phase was reduced, was prepared.

Specifically, when only the rutile phase was reduced, 14 mg of metallic Li particles were dissolved in 20 ml of ethylenediamine, so as to form a 1 mmol/ml solvated electron solution.

The 200 mg of dried TiO₂ nanocrystals (anatase, size: 25 nm or less, rutile, size: 140 nm or less, P25, size: 20 nm to 40 nm) were added and stirred for seven days. The reaction was carried out under closed and anhydrous conditions.

Then, 1 mol/L HCl was slowly added dropwise to the mixture to quench excess electrons and form a Li salt.

Lastly, the resulting composite was rinsed several times with deionized water and dried in a vacuum oven at room temperature, so as to obtain reduced titanium dioxide (Blue TiO₂).

When only the anatase phase was reduced, 14 mg of metallic Na particles were dissolved in 20 ml of ethylenediamine, so as to form a 1 mmol/ml solvated electron solution. The following treatment may be the same as in the case where only the rutile phase is reduced.

### [Example 1] HEPA Filter with Attached Photocatalyst to which CMC is Applied

First, photocatalyst powder was prepared by mixing a titanium dioxide (Blue TiO₂) photocatalyst having the rutile phase selectively reduced and tungsten trioxide (WO₃) as nano-sized powders at a mass ratio of 1:2.9 in a mortar.

Then, carboxymethyl cellulose (CMC) having a weight average molecular weight of 90K at 1% mass ratio was added to 100 mg of distilled water and added and dispersed in an ultrasonic washing machine, so as to prepare a CMC solution (mixed at a rate of 1 g of CMC having a weight average molecular weight of 90 K per 100 ml of distilled water).

Subsequently, the photocatalyst powder was added to the CMC solution and put into an ultrasonic washing machine, so as to evenly disperse for one hour.

After that, the HEPA filter was placed on a hot plate, and heated to 85° C. to 95° C., after which a solution containing the photocatalyst powder and CMC was dropped onto the HEPA filter. When the solution was dropped on the HEPA filter, the HEPA filter absorbed the solution, and when the solvent was all evaporated, the above process was repeated until all the solution was used up.

Then, the temperature of the HEPA filter was cooled to room temperature, so as to prepare a filter having a photocatalyst attached thereto according to the present application.

### [Comparative Example 1] HEPA Filter

A general HEPA filter without a photocatalyst attached thereto was used as Comparative Example 1.

### [Comparative Example 2] HEPA Filter with Attached Photocatalyst to which CMC is not Applied

First, photocatalyst powder was prepared by mixing a titanium dioxide (Blue TiO₂) photocatalyst in which only one of the anatase phase and the rutile phase was reduced and tungsten trioxide (WO₃) as nano-sized powders at a mass ratio of 1:2.9 in a mortar.

Subsequently, the photocatalyst powder was added to 100 mg of distilled water and put into an ultrasonic washing machine, so as to evenly disperse for one hour.

After that, the HEPA filter was placed on a hot plate, and heated to 85° C. to 95° C., after which a solution having the photocatalyst powder dispersed therein was dropped onto the HEPA filter. When the solution was dropped on the HEPA filter, the HEPA filter absorbed the solution, and when the solvent was all evaporated, the above process was repeated until all the solution was used up.

Then, the temperature of the HEPA filter was cooled to room temperature, so as to prepare a filter having a photocatalyst attached thereto.

### [Experimental Example 1] Comparison of HEPA Filters Before and After Attaching Photocatalyst

FIG. 3 is a digital camera picture and an optical microscope picture of filters according to one example and a comparative example of the present application. Specifically, an upper picture is a picture of a digital camera, and a lower picture is a picture of an optical microscope.

Referring to FIG. 3, the HEPA filter having a network structure can be confirmed from the digital camera picture (up) of Comparative Example 1, which is the HEPA filter before attaching the photocatalyst, and it can be confirmed that opaque colored polyethylene terephthalate (PET) stems are present. A shape can be confirmed more clearly from the optical microscope picture (down), and it can be confirmed that the surface is maintained in a smooth state.

Meanwhile, it can be confirmed from the digital camera picture (up) of Example 1, which is the HEPA filter having the photocatalyst attached thereto, that the color of the photocatalyst attached to the surface is weakly applied, and a clear surface change can be observed from the optical microscope picture (down). It can be observed that the photocatalyst is evenly distributed on the PET stem, and it can be also observed from the inset of the optical microscope picture (down) that a surface is different when compared to the HEPA filter before attaching the photocatalyst.

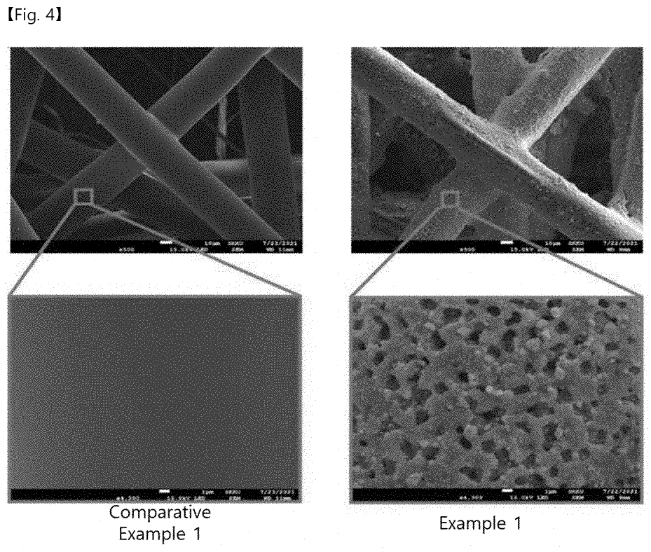

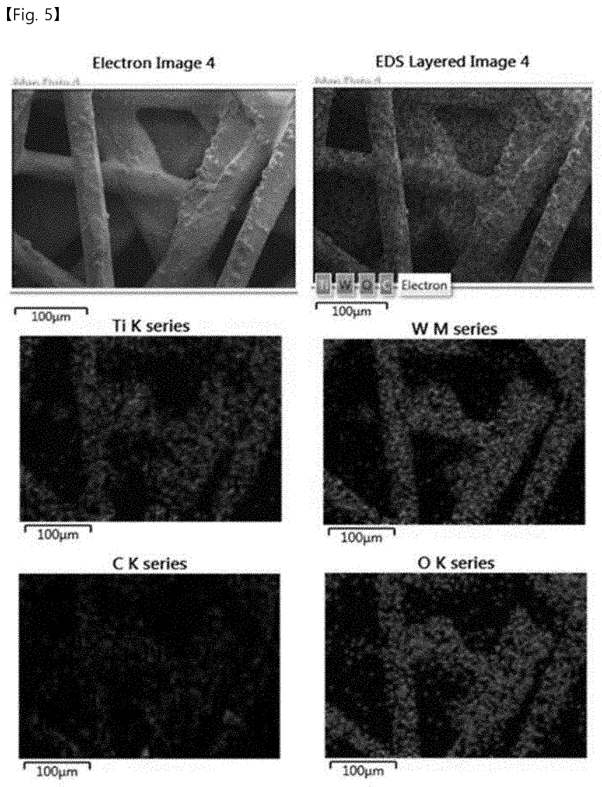

FIG. 4 is an SEM image of filters according to one example and a comparative example of the present application.

Referring to FIG. 4, it can be confirmed that Comparative Example 1 has a monotonous and smooth surface without any roughness. Meanwhile, it can be confirmed for Example 1 that many photocatalyst particles are attached to the surface, and it can be confirmed from the picture magnified 4300 times that there are many holes, and more organic compounds may be adsorbed due to this structure, and thus providing a potential increase in efficiency.

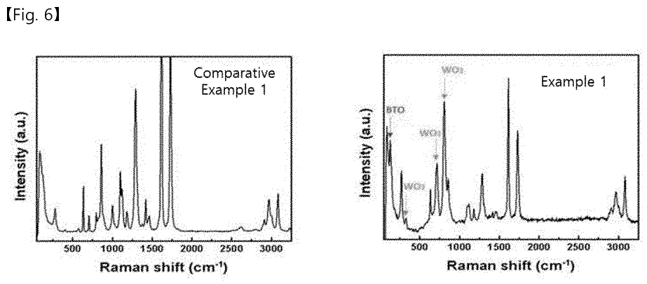

FIG. 5 is an SEM-EDS mapping image of filters according to one example of the present application.

Referring to FIG. 5, it can be confirmed that titanium (Ti), tungsten (W), carbon (C), and oxygen (O) are evenly distributed in the filter of Example 1.

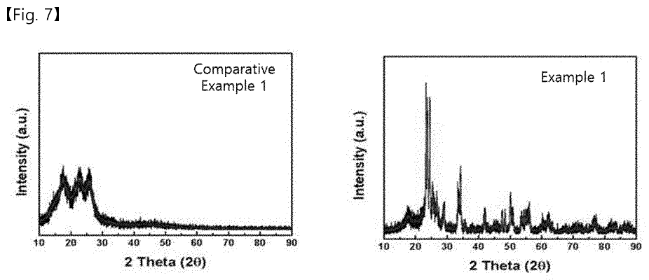

FIG. 6 is a Raman spectroscopic spectrum image of filters according to one example and a comparative example of the present application.

Referring to FIG. 6, a Raman spectrum can be confirmed with regard to the PET HEPA filter of Comparative Example 1, and a signal was detected at 1600 cm⁻¹, which may be attributed to a G signal due to a benzene structure. In the Raman spectrum of Example 1, signals of reduced titanium dioxide (blue TiO₂) and tungsten trioxide (WO₃) were detected along with the signals of the PET HEPA filter, indicating that the photocatalyst coexisted and was evenly distributed in the HEPA filter.

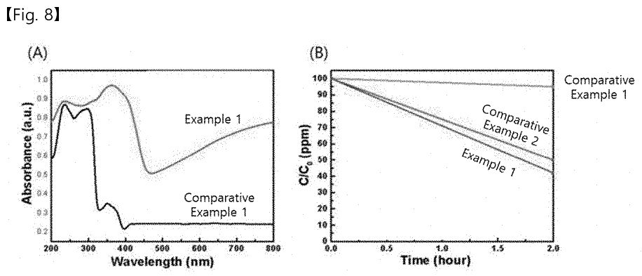

FIG. 7 is an X-ray diffraction spectroscopic spectrum image of filters according to one example and a comparative example of the present application.

Referring to FIG. 7, the X-ray diffraction spectroscopy spectrum of Comparative Example 1 shows the spectrum of the PET HEPA filter only, and it can be confirmed that a triplet was detected at 18, 23, and 26 degrees with the strongest signals, indicating the signal of the PET HEPA filter only.

Meanwhile, it can be confirmed from the X-ray diffraction spectrum of Example 1 that a large number of signals are detected along with the HEPA filter signal, because a signal of the reduced titanium dioxide (blue TiO₂) and a signal of the tungsten trioxide (WO₃) are simultaneously detected. Considering that the signal of the HEPA filter also coexists in the spectrum of Example 1, it is shown that the material state of the HEPA filter is maintained without any decomposition or damage even when the PET HEPA filter is heated at or above the glass transition temperature and cooled again, supporting that stability is maintained even after the process of manufacturing the filter having the photocatalyst attached thereto according to the present application.

### [Experimental Example 2] Measuring of Filter Effect after Attaching Photocatalyst

(A) of FIG. 8 is an ultraviolet-visible ray absorbance spectrum image of filters according to one example and a comparative example of the present application, (B) thereof is a graph of measuring an acetaldehyde reduction effect.

Referring to (A) of FIG. 8, it can be confirmed that Comparative Example 1, which is a general HEPA filter without a photocatalyst attached thereto, does not show any absorption in the visible ray region, but Example 1, which is the filter having the photocatalyst attached thereto, shows absorption in the visible ray region.

Referring to (B) of FIG. 8, it can be confirmed that the filter of Comparative Example 1 has no effect on reducing acetaldehyde, but Comparative Example 2 having the photocatalyst attached thereto shows an effect of 50% reduction, and the filter of Example 1 of the present application, to which the photocatalyst and CMC are applied together, shows an effect of 58%. These are all reduction effects under visible light, and show high efficiency not only in strong sunlight or ultraviolet light but also in general visible light.

### Experimental Example 3

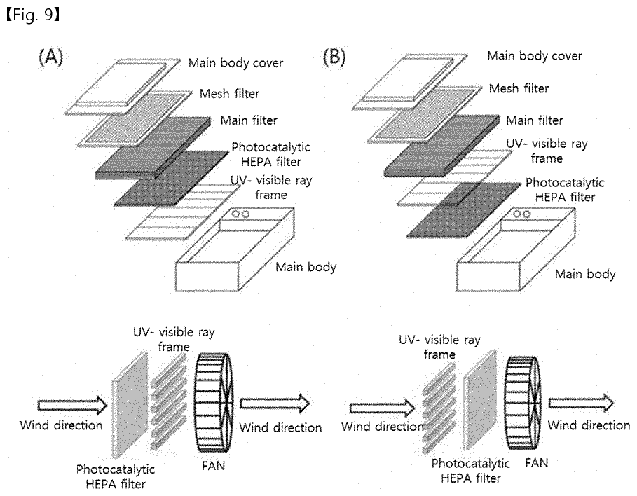

FIG. 9 is a schematic view of an air purifying device to which a filter according to one example of the present application is applied. Specifically, (A) of FIG. 9 shows a case in which the photocatalytic HEPA filter (Example 1) is disposed ahead of the UV-visible ray frame, and (B) shows a case in which the photocatalytic HEPA filter (Example 1) is placed behind the UV-visible ray frame.

Referring to (A) of FIG. 9, first, wind in the air is introduced into the main body by the FAN of the main body, passes through a main body cover, and passes through a mesh filter capable of filtering large particles. Then, the wind passes through a main filter which plays a main role of the air purifier, after which small particles and volatile organic compounds not collected in the main filter pass through the photocatalytic HEPA filter. At this time, the photocatalytic HEPA filter not only collects small particles, but also adsorbs organic matter by the photocatalyst, and the light emitted from an ultraviolet-visible ray frame disposed behind the photocatalyst HEPA filter enables the photocatalyst to decompose the organic matter. In a short time, the volatile organic compounds are decomposed, and the decomposed organic materials (safe for the human body) are introduced along the main body, and the air with volatile organic compounds removed is sprayed into the air again.

Referring to (B) of FIG. 9, all processes are the same as those of (A) of FIG. 9, except that the volatile organic compounds pass through the main filter, passes through the ultraviolet-visible light frame first, and then enters the photocatalytic HEPA filter.

Regarding the arrangement of (A) and (B) of FIG. 9, the effect of decomposing volatile organic compounds is the same, but in product application, it is possible to configure the structure of the product with applicable arrangement depending on whether a wire for supplying power to ultraviolet-visible rays is on the main body side or the main body cover side. In addition to changing the arrangement according to the power supply, a more efficient arrangement may be applied to the product in terms of product configuration.

### Experimental Example 4

An experiment was conducted to compare the performance of filters having the photocatalyst attached thereto according to the presence or absence of a hydrophilic polymer binder. Specifically, the filter of Example 1, which includes a photocatalyst and CMC as a hydrophilic binder, and the filter of Comparative Example 2, which includes only a photocatalyst, were compared with each other.

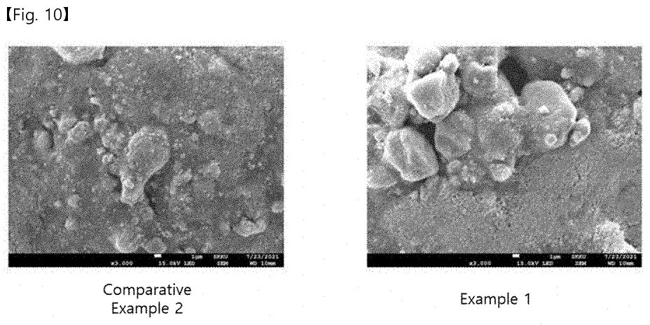

FIG. 10 is an SEM image of filters according to one example and a comparative example of the present application.

Referring to FIG. 10, it can be confirmed that Example 1 having CMC applied thereto has more aggregates formed compared to Comparative Example 2 without CMC applied thereto.

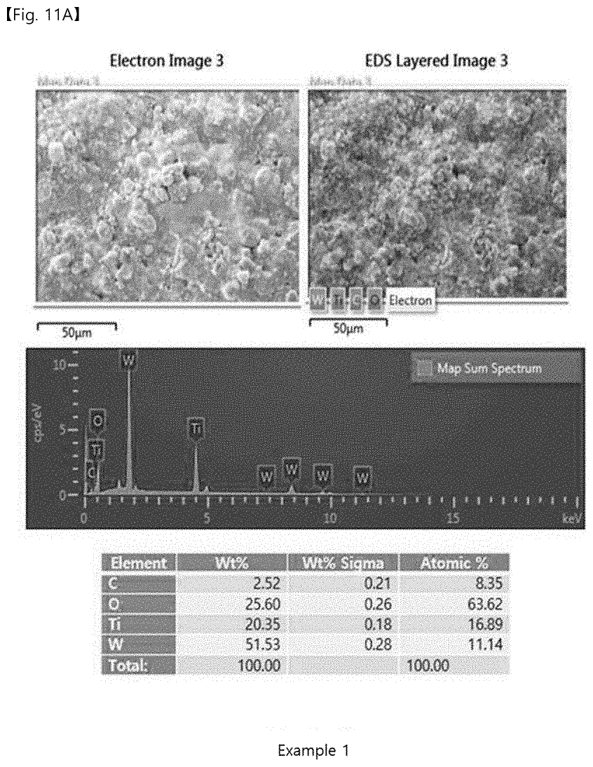

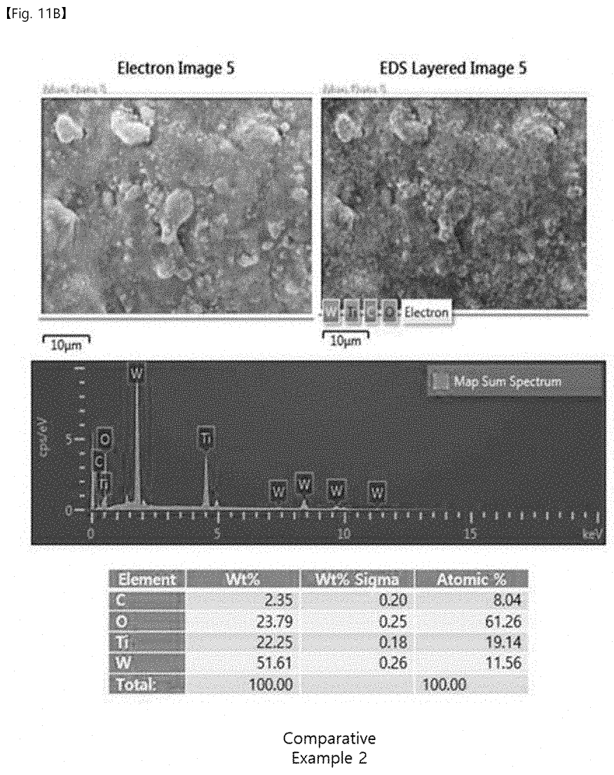

FIG. 11A and FIG. 11B are EDS analysis data of filters according to one example and a comparative example of the present application. Specifically, FIG. 11A is an EDS analysis data of a filter according to one Example 1 of the present application, and FIG. 11B is an EDS data of a filter according to one Comparative Example 2 of the present application.

Referring to FIG. 11A and FIG. 11B, it can be seen that Example 1 having CMC applied thereto has a higher ratio of carbon and oxygen than Comparative Example 2 without CMC applied thereto, and that's because CMC is included.

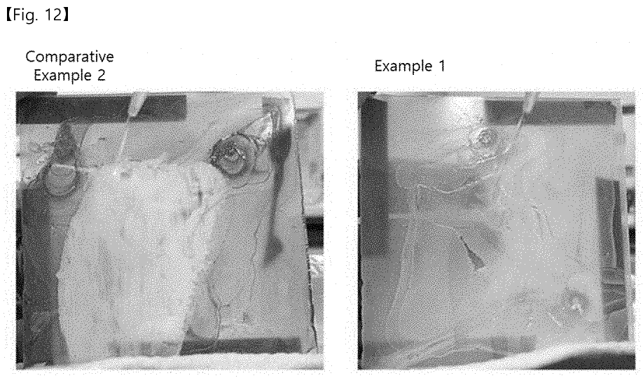

FIG. 12 is an experimental picture of comparing water resistance of filters according to an example and a comparative example of the present application.

Referring to FIG. 12, it can be seen that the photocatalyst was washed away when water was sprayed on the photocatalyst sample (Comparative Example 2) to which CMC was not added. Meanwhile, it can be confirmed that the photocatalyst was not washed away and had water resistance when water was sprayed on the sample (Example 1) to which CMC was added.

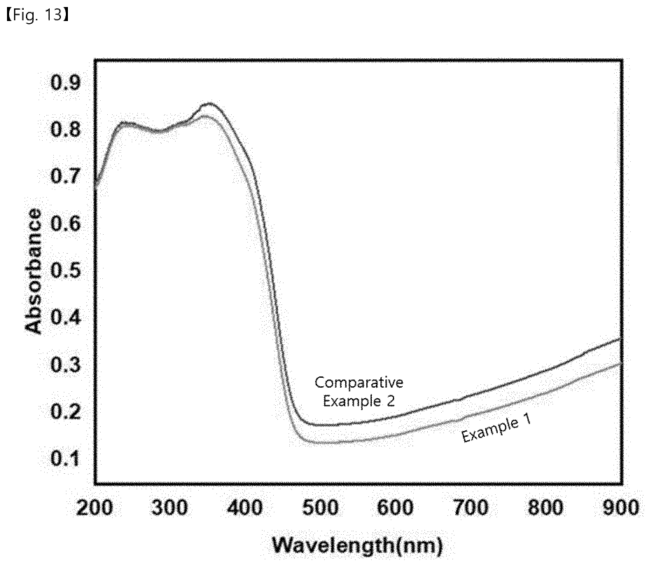

FIG. 13 is an UV/VIS absorption spectrum image of filters according to one example and a comparative example of the present application.

Referring to FIG. 13, it can be confirmed that the application of CMC does not negatively affect the efficiency of the photocatalyst, considering that there is no significant difference in the absorption spectrum depending on the application of CMC.

Through Experimental Example 4, it could be confirmed that attaching the photocatalyst to the filter using CMC does not reduce the efficiency of the photocatalyst and enables a filter with improved water resistance to be manufactured.

### Experimental Example 5

According to Example 1, a photocatalyst powder prepared by mixing titanium dioxide having the rutile phase selectively reduced and tungsten trioxide was prepared, and as shown in [Table 1] below, the molecular weight of carboxymethylcellulose (CMC) and the content of carboxymethylcellulose (CMC) were controlled, and the catalyst properties were evaluated.

Specifically, 300 mg of photocatalyst powder was prepared by mixing titanium dioxide having the rutile phase selectively reduced and tungsten trioxide in a weight ratio of 1:2.9. A CMC solution in which 1 wt % and 0.1 wt % of carboxymethylcellulose (CMC) having an weight average molecular weight of 90 K and 700 K were mixed per 100 ml of distilled water was prepared. The CMC solution was added to 8 ml of ethanol and mixed with 300 mg of photocatalyst powder, controlling the CMC solution to 0-1,000 ul, and preparing a HEPA filter as in Example 1. After that, the decomposition efficiency of acetaldehyde was measured for two hours under visible light conditions.

TABLE 1

| | | | | | | | | |
|---|---|---|---|---|---|---|---|---|
| CMC/ | 0 | 10 | 25 | 50 | 100 | 200 | 500 | 1000 |
| amount | | | | | | | | |
| (ul) | | | | | | | | |
| M = 90 k | 60% | 90% | 95% | 85% | 85% | 70% | 60% | 55% |
| 1 wt % | | | | | | | | |
| solution | | | | | | | | |
| M = 700 k | 60% | 85% | 95% | 95% | 80% | 65% | 60% | 50% |
| 0.1 wt % | | | | | | | | |
| solution | | | | | | | | |

As can be seen in [Table 1], it can be confirmed that when the amount of carboxymethyl cellulose (CMC) added is increased, adhesiveness increases, but when a large amount of carboxymethyl cellulose (CMC) is added, catalyst efficiency is lowered. Specifically, it can be confirmed that catalytic efficiency is remarkably high when 10 to 100 ul of the CMC solution is added, compared to when no CMC solution is added, and when more than 100 ul of the CMC solution is added.

### Experimental Example 6 and Experimental Example 7

As described in Experimental Example 5, 25 ul of CMC solution (weight average molecular weight of 90K, 1 wt %) was mixed with 300 mg of photocatalyst powder and 8 ml of ethanol, after which a process of dropping on the HEPA filter was repeated twice. (Experimental Example 6)

In addition, as described in Experimental Example 5, 300 mg of photocatalyst powder was mixed with 4 ml of ethanol and dropped onto the HEPA filter, after which 25 ul of CMC solution (weight average molecular weight of 90 K, 1 wt %) was mixed with 4 ml of ethanol and dropped onto the HEPA filter. (Experimental Example 7)

The HEPA filters prepared in Experimental Examples 6 and 7 were measured to have an acetaldehyde decomposition efficiency of 95%, substantially the same as that in Experimental Example 5 in which 25 ul of the CMC solution was added. In conclusion, it can be confirmed that providing the CMC solution and the photocatalyst powder solution alternately and repeatedly on the HEPA filter is an efficient method for providing a large amount of photocatalyst powder and a large amount of carboxymethylcellulose (CMC) to the HEPA filter.

### Experimental Example 8

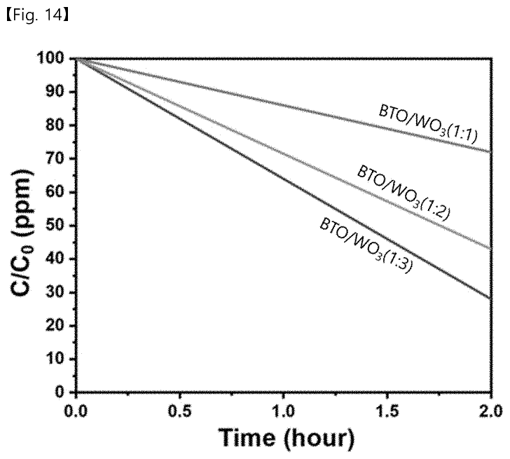

According to Example 1, with regard to the photocatalyst powder in which titanium dioxide having the rutile phase selectively reduced and tungsten trioxide are mixed, mixing was performed at ratios of 1:1, 1:2, 1:3, and 1:4; the photocatalyst powder and water were put in a 10 cm 10 cm Tedlar bag; oxygen gas was injected by bubbling water; and acetaldehyde was added to set a concentration to 100 ppm. After that, a change in the concentration of acetaldehyde for two hours under visible light conditions was confirmed as shown in FIG. 14.

When the ratio of titanium dioxide having the rutile phase selectively reduced and tungsten trioxide was 1:4, the concentration change was measured to be about 62% after two hours, and as shown in FIG. 14, the same was measured to be about 41% in the case of 1:2, and measured to be about 72% in the case of 1:1. In conclusion, when the mixing ratio of titanium dioxide having the rutile phase selectively reduced and tungsten trioxide was 1:3, it can be confirmed that catalytic efficiency is remarkably superior compared to the mixing ratios of 1:4, 1:2, and 1:1.

The above description of the present application is for illustrative purposes, and those skilled in the art will understand that it can be easily modified into other specific forms without changing the technical spirit or essential features of the present application. Therefore, the embodiments described above should be understood as illustrative in all respects and not limiting. For example, each component described as a single type may be implemented in a distributed manner, and similarly, components described as distributed may be implemented in a combined form.

The scope of the present application is indicated by the following claims rather than the detailed description above, and all changes or modifications derived from the meaning and scope of the claims and equivalent concepts thereof should be construed as being included in the scope of the present application.

What is claimed is:

1. A filter having a photocatalyst attached thereto comprising:
a substrate; and
a photocatalyst bonded on the substrate,
wherein the photocatalyst has each photocatalyst bonded and combined by a polymer binder, and the substrate and the photocatalyst are bonded by the polymer binder.

2. The filter of claim 1, wherein the polymer binder comprises a hydrophilic polymer.

3. The filter of claim 2, wherein the hydrophilic polymer comprises one selected from the group consisting of carboxymethyl cellulose (CMC), polyvinyl alcohol (PVA), polyvinylidene fluoride (PVDF), polytetrafluoroethylene (PTFE), polyamide, polyamine, styrene-butadiene rubber, and combinations thereof.

4. The filter of claim 1, wherein the photocatalyst comprises an anatase phase and a rutile phase, and comprises reduced titanium dioxide in which one of the anatase phase and the rutile phase is selectively reduced.

5. The filter of claim 4, wherein the reduced titanium dioxide is in the form of blue nanoparticles.

6. The filter of claim 4, wherein the photocatalyst further comprises one selected from the group consisting of WO₃, TiO₂ (anatase), TiO₂ (rutile), TiO₂, in which an anatase phase and a rutile phase are mixed, ZnO, CdS, ZrO₂, SnO₂, V₂O₃, and combinations thereof.

7. The filter of claim 1, wherein the substrate comprises one selected from the group consisting of polyethylene terephthalate, polyethylene, polypropylene, polycarbonate, polyvinyl chloride, polystyrene, concrete, glass, ceramic, metal, paper, wood, and combinations thereof.

8. A method for manufacturing a filter having a photocatalyst attached thereto, the method comprising:
dispersing a solution containing a photocatalyst and a polymer binder;
dropping the solution onto a substrate; and
cooling the substrate.

9. The method of claim 8, wherein the substrate is heated before performing the dropping, or is heated after performing the dropping.

10. The method of claim 9, wherein, when the substrate is a polymer compound, the heating is performed at or above a glass transition temperature of the polymer compound.

11. The method of claim 10, wherein the substrate heated at or above the glass transition temperature has the photocatalyst bonded by the cooling.

12. The method of claim 8, wherein, the polymer binder comprises a hydrophilic polymer.

13. The method of claim 8, wherein the dispersion is performed in a process selected from the group consisting of an ultrasonic process, a pulverization process by physical impact force, a pulverization process by physical shear force, a high-pressure process, a supercritical/subcritical process, and combinations thereof.

14. The method of claim 8, wherein the photocatalyst comprises an anatase phase and a rutile phase, and comprises reduced titanium dioxide in which one of the anatase phase and the rutile phase is selectively reduced.

15. An air purifying device comprising a filter having a photocatalyst attached thereto according to claim 1.
